(19) United States
(12) Patent Application Publication
(10) Pub. No.: US 2023/0397979 A1
(43) Pub. Date: Dec. 14, 2023

(54) MEDICAL IMPLANT FOR TREATING ANEURYSMS

(71) Applicant: Acandis GmbH, Pforzheim (DE)

(72) Inventors: Michael Büchert, Pforzheim (DE); Andreas Schüssler, Pfinztal (DE)

(21) Appl. No.: 18/033,757

(22) PCT Filed: Oct. 21, 2021
(86) PCT No.: PCT/EP2021/079204

§ 371 Date: Apr. 25, 2023

(30) Foreign Application Priority Data
Oct. 26, 2020 (DE) 10 2020 128 124.2

(51) Int. Cl. A61F 2/07

(52) U.S. Cl. CPC A61F 2/07; A61F 2002/068

(57) ABSTRACT

The disclosure relates to a medical implant for treating aneurysms, including a support structure, which has a compressible and expansible lattice structure lattice elements that define lattice openings, wherein the lattice structure is covered at least in part with an in particular electrospun membrane of fibres, which membrane includes at least one luminal functional layer and at least one abluminal protective layer, which each have pores, wherein the porosity of the functional layer is less than the porosity of the protective layer. The membrane is so configured that at least the pores of the functional layer open, as a result of a pressure gradient arising between a liquid pressure in an inner through channel of the support structure and a liquid pressure outside the protective layer, so as to increase the throughflow of liquid through the membrane.

The invention relates to a medical implant for the treatment of aneurysms in accordance with the preamble of patent claim 1. An implant of this type is known from EP 2 678 466 B1, for example.

EP 2 678 466 B1 concerns a stent for neurovascular applications which is covered with a nonwoven material. The nonwoven material is produced by electrospinning and comprises a plurality of layers, wherein an inner layer is liquid-tight and an outer layer is sponge-like in configuration. The inner layer serves to shield an aneurysm from a flow of blood in a blood vessel. The outer sponge-like layer is intended to enable endothelial cells and/or drugs to become embedded. The disadvantage with the known implant is that the implant can only be used for aneurysms which are distant from a branching blood vessel, from arteries or from arterioles. In the case of aneurysms, which are sited close to a vessel branch, when the known implant is used, there is a risk that the branching blood vessel will also be isolated from the bloodstream. This can result in a substantial deficiency in supply in regions of tissue, which are intended to be supplied with oxygen and nutrients by the branching blood vessel.

For this reason, the objective of the invention is to provide a medical implant for the treatment of aneurysms, which on the one hand enables an aneurysm to be covered efficiently, and on the other hand ensures that the blood supply in branching blood vessels continues to be ensured. This objective is achieved by means of the subject matter of patent claim 1.

Thus, the invention is based on the concept of providing a medical implant for the treatment of aneurysms, with a carrier structure, which has a compressible and expandable mesh structure with mesh elements, which delimit mesh openings. The mesh structure is covered, at least in sections, with a membrane formed or consisting of fibres. The membrane comprises at least one luminal functional layer and at least one abluminal support layer, which respectively have pores. The porosity of the functional layer in this regard is smaller than the porosity of the support layer. In accordance with the invention, the membrane is configured in a manner such that as a consequence of a pressure gradient which occurs between a liquid pressure in an inner through channel of the carrier structure and a liquid pressure outside the abluminal support layer, at least the pores of the luminal functional layer of the membrane open in order to increase the flow of liquid through the membrane.

Thus, the invention is based on the idea of allowing the membrane to act intelligently, so that it is then more permeable to liquid when a pressure gradient arises between a liquid pressure in the inner through channel of the carrier structure and a liquid pressure outside the membrane. In the implanted state, the pores of the membrane open, in particular in the functional layer, when the membrane, which is installed with the implant in a main blood vessel bridges a branching blood vessel, and therefore a pressure gradient exists between the main blood vessel, in particular an artery, and an artery or arteriole, which branches off it. The blood flowing through the main vessel into the branching blood vessel, in particular into the branching artery or the branching arteriole, has a liquid pressure which forces the blood into the branching blood vessel or the branching arterioles. The inner membrane is therefore designed so that this liquid pressure is sufficient to open the pores of at least the functional layer of the membrane locally to an extent that at these sites at which blood vessels branch off, in particular arteries or arterioles, they are permeable to blood. In particular, the membrane is adapted in a manner such that it becomes permeable to blood, so that areas of tissue, which are in connection with the branching blood vessels or arterioles are sufficiently supplied with oxygen and nutrients.

In regions in which the implant covers an aneurysm, however, a pressure gradient of this type does not exist, so that the pores of the membrane remain closed, whereupon the aneurysm is efficiently shielded from the flow of blood inside the blood vessel. In any case, the shielding is more efficient than is the case with previously known or conventional flow diverter implants.

In this regard, the shielding does not have to be in the manner of a complete barrier to liquid. Rather, a severely reduced exchange of liquid between the blood inside the aneurysm and the blood inside the main vessel may persist. The shielding of the aneurysm is sufficient, however, to reduce the flow of blood inside the aneurysm to an extent that the blood inside the aneurysm coagulates, and therefore a thrombus forms inside the aneurysm. In this respect, the aneurysm naturally atrophies, wherein the membrane of the implant ensures that the thrombus does not leave the aneurysm. The formation of a thrombus reduces the pressure on the wall of the aneurysm so that the risk of rupture of an aneurysm and the associated blood loss or haemorrhagic stroke is reduced.

The advantage of the implant in accordance with the invention is obvious. In the treatment of aneurysms, in particular cerebral aneurysms, by means of the implant in accordance with the invention, it is not absolutely necessary for an operator to determine the position of the implant exactly. In particular, the operator does not have to take care to insert an implant of a specific length in order to avoid covering branching blood vessels. This is also particularly the case with smaller branching blood vessels, that are known as “perforating arteries”, which typically have a diameter of between 300 μm and 850 μm, and branching arterioles, which mostly have a diameter of between 50 μm and 300 μm. Moreover, it is possible to use a medical implant with a standard length for any type of aneurysm, because the implant enables blood to flow in branching blood vessels, in particular arteries or arterioles, even when the implant covers the branching blood vessel, in particular arteries and branching arterioles. This considerably speeds up and facilitates the treatment of aneurysms.

Preferably, the membrane comprises an electrospun textile, which forms the fibres. The textile is preferably multi-layered and is formed from fibres with different properties. In particular, the textile may comprise the support layer and the functional layer, which are respectively formed from fibres, wherein the fibres have different properties, in particular a different elasticity and/or fibre thickness. On the luminal side, i.e. the side facing the lumen of the main vessel, the membrane preferably has a relatively low porosity, which increases under a pressure gradient (and thus forms the actual functionality) and on the abluminal side, i.e. the side facing the vessel wall, it has a relatively higher porosity, which acts in a supportive manner. In this regard, the present application distinguishes between the luminal functional layer, which determines the functionality of the intelligently-acting membrane, and the abluminal support layer which supports the functional layer. The porosity of the support layer substantially does not vary, or hardly varies, under the influence of a pressure gradient generated by a branching blood vessel or a branching arteriole. In addition, the support layer may be designed in a manner such that it prevents the fibres of the functional layer from shifting in the radial direction.

The functional layer and the support layer may in particular differ in the type of arrangement of the fibres. Preferably, the support layer comprises or consists of fibres, which are primarily interconnected and therefore form a stable layer. The freedom of movement of the fibres of the support layer is limited by the interconnections. On the other hand, the functional layer may be formed from or comprise fibres, which primarily lie loosely on top of one another. This permits a higher freedom of movement of the fibres, so that larger pores can be formed by displacement of the fibres. Preferably, the fibres of the support layer have a relatively larger diameter and a relatively higher strength or Shore hardness, whereas the fibres of the functional layer may have a relatively smaller diameter and a relatively lower strength or Shore hardness.

In principle, the functional layer and the support layer may form layers, which are clearly delimited from each other. However, it is possible for the functional layer and the support layer not to be clearly delimited from each other, but for these to be regions of different porosities, which merge seamlessly with one another. Preferably, the membrane forms a compromise between functionality (opening or non-opening of pores by movement or deformation of the fibres) and support action. In this regard, the regions with differing porosities, in particular the functional layer and the support layer, may blur into each other that the functional layer and the support layer cannot be distinguished one from the other in the direction of the pressure gradient. Moreover, the membrane may have a near-identical porosity overall in the direction of the pressure gradient. This is the case for the unloaded state of the membrane, i.e. without the influence of the pressure gradient. In contrast, under the influence of the pressure gradient, some of the fibres of the functional layer of the membrane move or deform, so that larger pores are formed and therefore the membrane becomes sufficiently permeable to blood for branching blood vessels to be able to supply sufficient oxygen and nutrients to downstream regions of tissue. The deformation may be an elastic or plastic deformation. In any case, the generation of the larger pores occurs without rupture of the fibres. Moreover, the enlargement of the pores can occur without destruction and equally can be reversed in a non-destructive manner, for example when the pressure gradient between the liquid pressure in the inner through channel of the carrier structure and the liquid pressure outside the support layer reduces.

Preferably, the functional layer and the support layer are adhesively bonded to each other. In particular, individual fibres of the functional layer and of the support layer cross over or under each other, so that a coherent membrane is formed. The luminal functional layer and the abluminal support layer preferably differ in their porosity and the function associated therewith. While the abluminal support layer is substantially intended to stabilize the membrane overall and to hold the fibres of the luminal functional layer in their specified position, the functional layer serves to shield an aneurysm efficiently from a bloodstream. At the same time, however, the functional layer can open up for blood to flow into a branching blood vessel in order to ensure the supply of blood to downstream regions of tissue.

Preferably, the membrane comprises a functional layer and a support layer. However, this does not exclude the fact that the membrane could also have other layers, for example two or more functional layers and/or two or more support layers and/or further other layers. Furthermore, the membrane may have a coating, for example a coating with anti-thrombogenic properties. This coating is provided in a manner such that the individual fibres of the membrane are sheathed with the coating.

In order to ensure passage through the functional layer at sites at which a blood vessel branches off from the main vessel into which the implant has been inserted, in a preferred variation of the invention, the fibres of the membrane, in particular at least the fibres of the functional layer, are arranged loosely on top of one another at points of intersection, so that intersecting fibres are movable with respect to each other at the points of intersection. In other words, the intersecting fibres of the inner membrane can slide over one another, whereupon the pores, which are delimited by the fibres can open as a consequence of the aforementioned pressure gradient. Thus, a perfusion region can be generated, through which blood can be directed into a branching blood vessel.

As an alternative or in addition, the fibres of the membrane, in particular at least the fibres of the functional layer, may be elastically and/or plastically deformable in order to deflect as a consequence of a pressure gradient and to form enlarged pores so that locally, a flow of blood into a branching blood vessel, in particular an artery or a branching arteriole, can be obtained which is sufficient to supply blood to downstream regions of tissue.

For the advantageous function in accordance with the invention, i.e. that the luminal functional layer can open in sections or locally for the perfusion of blood or liquid, it is advantageous for the functional layer to have a high flexibility. In particular, the fibres of the functional layer should be as flexible as possible in order to permit deformation, which leads to an enlargement of the pores of the functional layer. The enlargement of the pores advantageously occurs without destruction or without the formation of ruptures in the fibres. In this regard, it is preferable for the thickness of the fibres of the functional layer to be particularly small. In particular, the fibres may have a thickness of less than 500 nm, in particular at most 400 nm, in particular at most 300 nm, in particular at most 200 nm, in particular at most 100 nm. In contrast, the abluminal support layer, which has a stabilizing function, should comprise more stable fibres. This can be obtained by providing the fibres of the support layer with a fibre thickness of at least 500 nm, in particular at least 750 nm, in particular at least 1000 nm, in particular at least 1250 nm, in particular at least 1500 nm.

In order for the functional layer to function by enabling the flow of blood in branching blood vessels, but at the same time to shield an aneurysm efficiently from the flow of blood in the main vessel, in a preferred embodiment of the medical implant, the functional layer has a thickness of at most, in particular less than 10 μm, in particular at most 8 μm, in particular at most 6 μm, in particular at most 4 μm, in particular at most 2 μm. Correspondingly, it contributes to the stabilizing function of the support layer when it has a thickness of at least 3 μm, in particular at least 5 μm, in particular at least 6 μm, in particular at least 7 μm, in particular at least 8 μm.

In order to be able to shield an aneurysm efficiently, a particularly low porosity for the functional layer is advantageous. In this regard, in a preferred variation of the invention, the functional layer has a porosity of less than 50%, in particular at most 40%, in particular at most 30%. In contrast, the support layer, which should be permanently permeable to blood, may have a porosity of at least 50%, in particular at least 60%, in particular at least 70%, in particular at least 80%, in particular at least 90%. In the context of the present application, “porosity” should be understood to mean the ratio between the open surface area of a tissue, i.e. the sum of the surface areas of all of the pores, and the total surface area of that tissue.

Particularly preferred is a variation of the medical implant, in which the functional layer comprises at least 10 pores which have an inscribed circle diameter of at most 10 μm, in particular at most 8 μm, in particular at most 6 μm, in particular at most 4 μm, in particular at most 2 μm, in particular at most 1 μm, over a surface area of 100000 μm². As an alternative or in addition, the support layer may comprise at least 5 pores, in particular at least 10 pores, which have an inscribed circle diameter of at least, in particular more than, 10 μm, in particular at least 15 μm, in particular at least 20 μm, in particular at least 25 μm, in particular at least 30 μm, in particular at least 40 μm, in particular at least 50 μm, in particular at least 60 μm over a surface area of 100000 μm². Having regard to the different functions of the functional layer and of the support layer, wherein the functional layer should have a flexibility which enables a perfusion of blood in the case of an appropriate pressure gradient, and on the other hand the support layer is intended to stabilize the functional layer so that it is not released from the carrier structure, advantageously, the thickness of the fibres of the functional layer is smaller than the fibres of the support layer.

In this regard, preferably again, the functional layer or its fibres has/have a higher ductility than the support layer or its fibres.

The fibres of the functional layer may be formed from a material, which has a lower Shore hardness than the material of the fibres of the support layer. In particular, the material of the fibres of the functional layer may have a Shore hardness of at most 90A, in particular at most 80A, in particular at most 70A, in particular at most 60A, in particular at most 50A, and/or the material of the fibres of the support layer may have a Shore hardness of at least 90A, in particular at least 100A, in particular at least 60D, in particular at least 70D, in particular at least 80D.

The membrane, in particular the functional layer and the support layer, may comprise a thermoplastic polyurethane. This does not exclude the possibility that the functional layer and/or the support layer could respectively comprise other plastic materials. In a preferred variation, however, the functional layer and the support layer consist of a thermoplastic polyurethane. It is also possible for the functional layer or the support layer, in particular the functional layer and the support layer or the membrane overall, to be formed from an absorbable or resorbable material. In this regard, it may be possible for the functional layer and/or the support layer to dissolve over a specific period of time by contact with blood and therefore after this period of time, only the carrier structure will remain in the blood vessel. Preferably, the absorbable or resorbable material is selected or adapted in a manner such that it dissolves after or within a period of time within which the aneurysm shielded by the functional layer will atrophy. In other words, the functional layer and/or the support layer should not dissolve until the aneurysm has atrophied.

The fibres of the functional layer may also be configured as concentric fibres. Such concentric fibres comprise a fibre core and a fibre sheath. The fibre core is preferably formed from a softer material than the fibre sheath, wherein the thickness of the fibre sheath is smaller than the fibre core. The fibre sheath may comprise a relatively harder material. In this manner, high flexibility of the individual fibres can be achieved via the fibre core, so that the fibres can deform well in order to open up the pores as a consequence of the influence of a pressure gradient on the membrane. In this regard, the fibre core may comprise a material with a Shore hardness of at most 90A, in particular at most 80A, in particular at most 70A, in particular at most 60A, in particular at most 50A, and/or the material of the fibre sheath may have a Shore hardness of more than 90A, in particular at least 100A, in particular at least 60D, in particular at least 70D, in particular at least 80D. The relatively harder material of the fibre sheath, on the other hand, serves to enable the fibres to slide on one another correctly, so that good enlargement of the pores is also obtained via the displacement of the fibres.

In a preferred variation of the invention, the membrane may extend around the entire circumference of the carrier structure. However, a membrane which extends only partially around the circumference of the carrier structure may also be conceivable, for example to enable blood to be supplied from the supplying blood vessel to the two branching vessels in the case of bifurcated aneurysms. The preferred variation in which the membrane extends around the entirety of the carrier structure, has particular advantages, however. On the one hand, series production of the medical implant is particularly simple using this type of variation. On the other hand, the membrane which is closed in the circumferential direction is self-stabilizing, so that the membrane will definitely adhere to the carrier structure and will not be released from the carrier structure. In particular, the carrier structure can carry out its stabilizing function particularly efficiently in this manner.

Different variations are conceivable regarding the carrier structure. On the one hand, the carrier structure may be monolithic in configuration, wherein the mesh elements of the mesh structure form webs which delimit mesh openings of the mesh structure which are formed as cells. In other words, the carrier structure may have a mesh structure, which is cut from a tubular starting material. This may be carried out by laser cutting, for example. Cutting the tubular starting material produces webs which delimit cells. On the other hand, the carrier structure may also have interwoven wires, wherein the wires form the mesh elements of the mesh structure and delimit mesh openings of the mesh structure which are formed as interstices. In this variation, the carrier structure therefore has a network of interwoven wires which form the mesh structure. The wires cross over and under each other, whereupon interstices are formed between the wires which cross over and under each other. It is also possible for the carrier structure to consist of wire elements or to comprise wire elements which do not intersect (wire forming). Moreover, the wire elements may be arranged in a common circumferential plane and be connected together, for example by spot welding. It is also possible for the monolithic configuration of the carrier structure to be produced by a combination of lithography and a sputter process, for example physical vapour deposition (PVD), in particular by magnetron sputtering.

Advantageously for the usage of the medical implant in accordance with the invention, it has good bending flexibility and can expand well from a compressed state, which is as small as possible to an expanded state which is as large as possible. This expansion may preferably occur automatically by using appropriate superelastic materials, for example shape memory alloys. In this regard, in particular, the carrier structure may be self-expandable. The implant may also comprise or consist of shape memory plastics.

Although in a preferred embodiment of the invention the mesh elements of the carrier structure comprise or consist of a self-expandable shape memory alloy such as nitinol, it is also conceivable to produce the carrier structure from balloon-expandable materials such as stainless steel or CoCr alloys. The latter is then in particular appropriate if the carrier structure is particularly short in length and/or is intended to have a particularly high radial force.

Good flexibility of the implant, both as regards the bending flexibility and also as regards the expansion capability, can be achieved by adjusting the ratio between the total layer thickness of the membrane and the height of the mesh elements or the wall thickness of the carrier structure appropriately. In a particularly preferred variation, the thickness of the membrane is at most 40%, in particular at most 30%, in particular at most 20%, in particular at most 10% of the height of the mesh elements, in particular the webs or the wires.

In a carrier structure, which is monolithic in configuration, the height of the mesh elements corresponds to the height of the webs or the wall thickness of the carrier structure. In the case of a carrier structure, which is formed from interwoven wires, the height of the mesh elements corresponds to the wire thickness. The total wall thickness of the carrier structure differs from this because the wires cross over each other at points so that the wall thickness of the carrier structure is twice the height of the mesh elements, i.e. the wire diameter. In any case, the total layer thickness of the membrane is limited in order to ensure that the membrane has a high flexibility in order to be able to correctly follow a curvature or expansion of the carrier structure.

It is further advantageous for the overall flexibility of the implant when, as is preferably the case, the height of the mesh elements, in particular the webs or the wires, is between 40 μm and 160 μm, in particular between 40 μm and 150 μm, in particular between 40 μm and 130 μm, in particular between 40 μm and 110 μm, in particular between 40 μm and 100 μm, in particular between 50 μm and 90 μm, in particular between 50 μm and 80 μm.

In general, in preferred variations of the implant in accordance with the invention, it can be specified that a ratio between the thickness of the membrane and the height of the mesh elements, in particular the webs or the wires, is at most 1/3, in particular at most 1/4, in particular at most 1/5, in particular at most 1/8, in particular at most 1/10, in particular at most 1/12, in particular at most 1/15, in particular at most 1/20. In other words, the height of the mesh elements is preferably two times to ten times, in particular 3 times to 8 times, in particular 4 times to 6 times the total thickness of the membrane.

More advantageously, the implant is easily visible under radiographic monitoring. This makes it easier for the operator to determine the position of the implant in the blood vessel and to monitor whether the membrane is carrying out its function (shielding an aneurysm but at the same time allowing good perfusion of branching blood vessels or arterioles). In this regard, the implant may be provided with radiopaque materials, at least in parts or locally. Such materials may be gold, platinum or tantalum, as well as alloys thereof.

As an example, radiographic markers, for example in the form of rings, coils or sleeves, may be arranged at the longitudinal ends of the carrier structure. In particular, three radiographic markers per longitudinal end are advantageous in order to make the implant discernible. In addition or as an alternative, it is also possible to arrange such radiographic markers in a central region of the carrier structure. In addition, additional filaments which are more radiopaque may be woven into the carrier structure. In particular, a filament of this type may be wound around a row of mesh elements which are mutually aligned. A filament of this type may be formed from what is known as DFT wire (drawn filled tube) wire.

Furthermore, it is conceivable that at least individual mesh elements of the carrier structure may comprise a radiopaque core material which is sheathed with a shape memory material (DFT wire). Similarly, at least individual, preferably all of the fibres of the membrane may be provided with a radiopaque core material and a sheath of another material, for example a polyurethane. In order to improve the radiopaque visibility, radiopaque materials may also be arranged between the functional layer and the support layer. Thus, for example, at least one radiopaque nonwoven material or at least one radiopaque strip may be arranged between the functional layer and the support layer. Finally, it is also possible to apply the radiopaque material, in particular tantalum, niobium, platinum or gold, to the carrier structure with the aid of sputter technology (in particular by magnetron sputtering) or to integrate it into it. The fibres may also be formed from a plastic blended with a radiopaque material. Thus, for example, a plastic may be blended with at least 20% barium sulphate, so that the membrane is visible at least in still radiographic images.

In a further embodiment of the invention, the implant is provided with an anti-thrombogenic coating so that each fibre of the membrane is surrounded by this coating. A coating of this type has the advantage that the pores of the membrane, which are generated by the movement of the fibres of the membrane, remain open and are not closed by the deposition of blood platelets.

Preferably, the coating has a layer thickness of at most 10 nm. The coating may comprise fibrin and/or heparin. In particular, the coating may comprise heparin covalently bonded to fibrin. A coating of this type is described in the Applicant's document DE 10 2018 110 591 A1, to which reference should be made in respect of the composition of the coating.

In general, the membrane described here (as part of the implant in accordance with the invention) may not only be of application to the treatment of aneurysms, but also for other cerebrovascular diseases. An example in this regard is the treatment of arteriovenous malformations (known as AVMs) and arteriovenous fistulae. In this regard, the porosity and flexibility of the membrane may be adjusted in a manner such that the arteriovenous short-circuit still supplies the veins with blood, but the inflow and therefore the pressure rise in the veins no longer leads to rupture thereof.

The membrane as a whole may be produced by an electrospinning process. As an example, the functional layer and the support layer may be produced independently of each other by electrospinning and then joined together on a carrier structure. Because of the nonwoven-like structure of the electrospun functional layer and of the electrospun support layer, the two layers connect together to form a coherent membrane, because fibres of the support layer are applied directly to the fibres of the functional layer by the electrospinning and are therefore adhesively bonded together. The process parameters and/or the materials for the production of the functional layer and of the support layer differ in order to fulfil the different functions of the functional layer and the support layer. In addition, it is also possible for the functional layer and the support layer to be produced in a common production step. To this end, an electrospinning process may be used in which different materials are deposited at the same time so that the membrane, which is generated directly functions on the one hand as the functional layer and functions on the other hand as the support layer. In general, the aim is for the overall thickness of the membrane, and also the thicknesses of the functional layer and the support layer, to be substantially constant over the length of the membrane. However, it may also be advantageous for the aforementioned thicknesses to very over the length of the membrane.

In a preferred further embodiment of the invention, the functional layer has a perforation in the region of mesh openings. The perforation may in particular comprise holes, straight slits, curved slits and/or T-shaped slits. By means of the perforation, openings are produced in the functional layer, which facilitate local opening of the functional layer in the case of a pressure difference between the liquid pressure in the inner through channel of the carrier structure and the liquid pressure outside the support layer. The support layer, which is arranged above it, limits the opening process, so that the opening or perforation does not expand too far. This ensures that opening occurs even with low pressure gradients. In this regard, the perforation is preferably configured in a manner such that a local opening of the functional layer occurs in regions, which are subjected to a pressure gradient, which usually occurs between the main blood vessel and a branching blood vessel or a perforating vessel. In particular, the perforation is set so that opening of the functional layer does not take place when a smaller pressure gradient exists, for example a pressure gradient, which usually occurs between a main blood vessel and an aneurysm. However, a local opening of the functional layer is intended to occur in order to allow blood to flow in a perforating vessel.

The perforation may be produced by laser processing of the membrane, in particular of the functional layer, and/or by solvent spraying. In the case of laser processing, the functional layer in particular may be provided with a perforation pattern by means of a UV laser or a femtosecond or picosecond infrared laser. Particular perforation patterns, which may be considered are holes, straight slits or cuts, gill-like, curved slits and/or T-shaped slits or cuts. Distributing the perforations over a larger region of the membrane in a pattern is generally preferred. Upon insertion of the implant, it is ensured that a perforation is positioned in front of a possibly covered side vessel and therefore the passage of liquid through the membrane into the side vessel is made possible.

In the case of solvent spraying, a mist of fine droplets of a solvent or solvent-polymer mixture of a defined size is produced. When the mist impinges upon the functional layer, the fibres of the functional layer are dissolved, and therefore holes are formed in the functional layer.

Furthermore, it may be conceivable that masking during the production of the membrane, in particular the functional layer, may produce a perforation. Thus, the functional layer may be masked in a manner such that during the formation of the functional layer by means of a spray process, a perforation pattern is generated or preserved. This production variation is particularly suitable for the formation of holes as the perforation pattern.

The medical implant in accordance with the invention may preferably be produced by a method, which has the following steps:
a. providing the carrier structure;
b. applying the functional layer to the carrier structure;
c. perforating the functional layer by a laser cutting process or by solvent spraying; and
d. applying the support layer to the functional layer.

As an alternative to forming the perforations by means of a laser cutting process or by means of solvent spraying, the functional layer may also be provided with the perforation by leaving individual areas free when applying the functional layer. This may be carried out by using a mask, for example, which is placed on the carrier structure prior to spraying on the functional layer and is removed again after spraying on the functional layer.

The method described enables simple and efficient production of an implant with an intelligent membrane, regions of which can open up as a consequence of an appropriately high pressure gradient in order to enable a liquid to pass through.

Finally, a further support layer may be arranged between the carrier structure and the functional layer. The further support layer may have fibres with a fibre thickness which is greater than the fibre thickness of the fibres of the functional layer. Moreover, the density of the fibres of the support layer may be lower than the density of the fibres of the functional layer.

The invention is described in more detail with the aid of exemplary embodiments and with reference to the accompanying schematic drawings, in which:

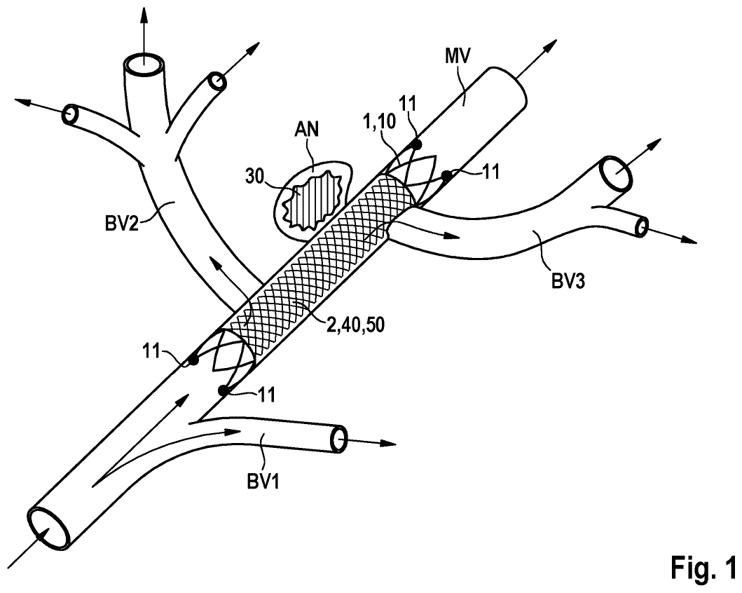

FIG. 1 shows a section of a blood vessel system into which a medical implant in accordance with the invention is inserted;

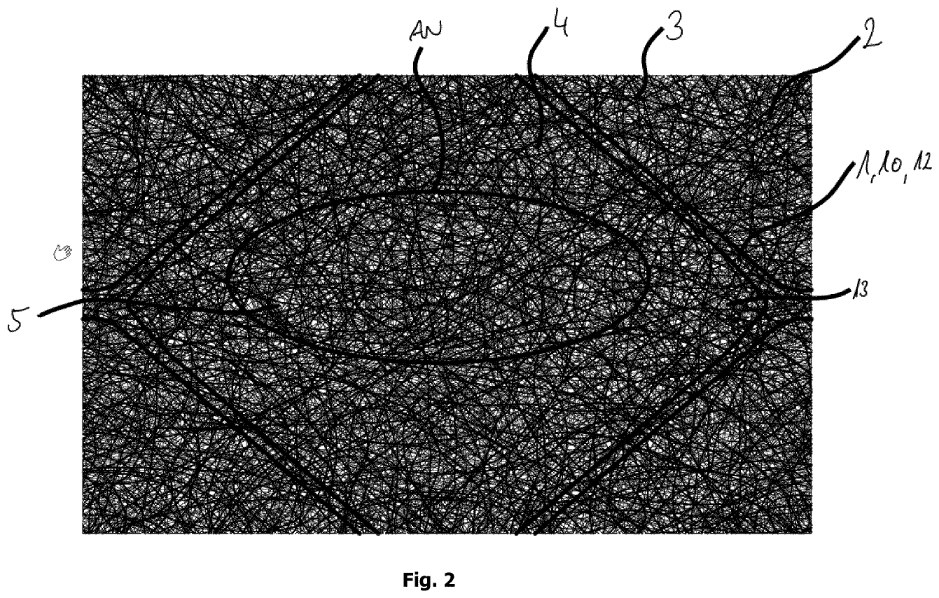

FIG. 2 shows a detailed section of the implant of FIG. 1 covering an aneurysm;

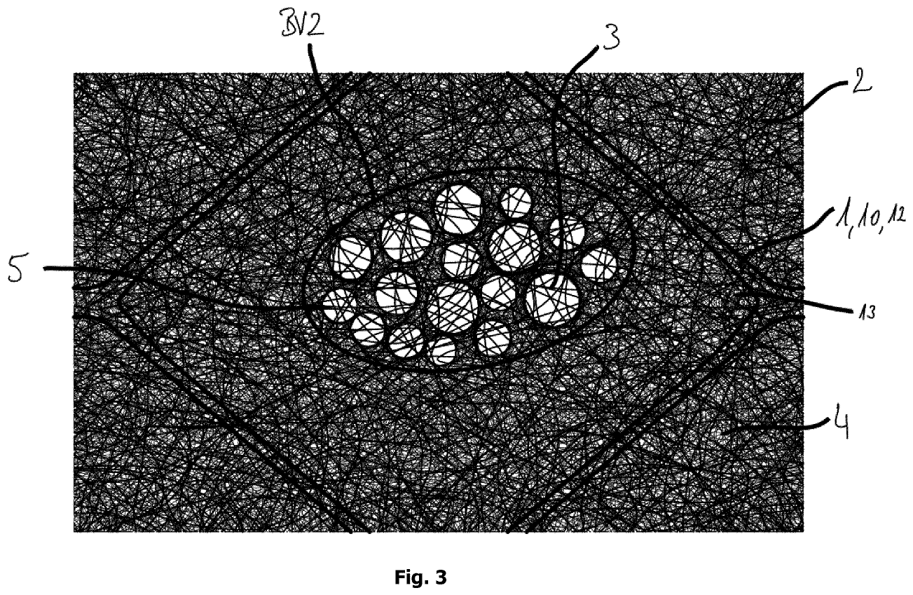

FIG. 3 shows a detailed section of the implant of FIG. 1 covering a branching blood vessel; and

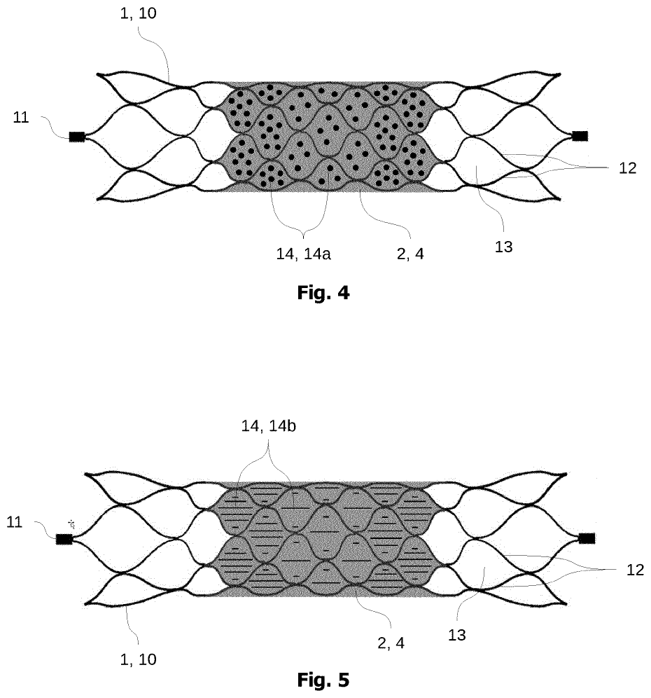

FIGS. 4 to 7 each show a side view of a medical implant in accordance with the invention in preferred exemplary embodiments, each showing a different perforation of the functional layer.

FIG. 1 shows a section of a blood vessel system with a main vessel MV and three side vessels BV1, BV2, BV3 branching from the main vessel MV. The main vessel MV also has an aneurysm AN, which is arranged between the second side vessel BV2 and the third side vessel BV3. In particular, the aneurysm is positioned close to the third side vessel BV3.

In order to treat the aneurysm AN, the medical implant in accordance with the invention is inserted. The medical implant comprises a carrier structure 1, which is formed by a mesh structure 10 with mesh elements. The mesh elements may be webs 12, which are interconnected into one piece and therefore form the mesh structure 10. In this regard, the webs 12 delimit cells 13 of the mesh structure 10. As an alternative, the mesh structure 10 may also be formed by interwoven wires. In order to make the mesh structure 10 or the carrier structure 1 visible for radiographic monitoring when inserting the implant into the blood vessel system or into the main vessel MV, radiographic markers 11 are provided on the respective longitudinal ends of the mesh structure 10. Preferably, a plurality of radiographic markers 11 are arranged at each longitudinal end of the mesh structure 10 and are positioned at regular distances in the circumferential direction of the mesh structure 10.

Furthermore, the implant has a membrane 2, which comprises a luminal functional layer 4 and an abluminal support layer 3. The functional layer 4 and the support layer 3 preferably overlap completely, and therefore have the same length in the longitudinal direction of the mesh structure 10. However, preferably, the support layer 3 protrudes beyond the functional layer 4, at least at the longitudinal ends, preferably by a few millimetres. As can be seen in FIG. 1, the mesh structure 10 may be longer than the membrane 2.

The implant is arranged in the main vessel MV in a manner such that the implant, in particular the membrane 2, completely covers the neck of the aneurysm AN. In addition, an embolization means 30 may be arranged in the aneurysm AN. Specifically, the medical implant may be supplied by itself or as a set together with an embolization means 30. The embolization means 30 may be a gel, for example. As an alternative, the embolization means 30 may also be formed by coils, i.e. chaotically twisted microwires. The embolization means 30 may be introduced into the aneurysm AN after the implant has been inserted into the main vessel MV. As an example, coils may be fed through the membrane 2 into the aneurysm via a microcatheter. The membrane 2 is or its fibres are so flexible in this regard that the microcatheter expands the pores of the membrane 2 and can therefore channel a path into the aneurysm AN.

As can also be seen in FIG. 1, the membrane 2 bridges not only the aneurysm AN, but also the second side vessel BV2 and the third side vessel BV3. This is where the benefits of the particular function of the membrane 2 take effect. The membrane 2 comprises the abluminal support layer 3, which has a larger porosity than the luminal functional layer 4. The support layer 3 in this regard is porous in a manner such that it is permanently permeable to blood. In contrast, the functional layer 4 is essentially of low permeability to blood, in particular semi-permeable, and above all less permeable to blood than the support layer 3. At the same time, however, the functional layer 4 is flexible in a manner such that with an appropriate application of force, it becomes permeable to blood or more permeable to blood. A required force of this type may be generated by the pressure gradient, which is set up between the blood pressure in the main vessel MV and the reducing blood pressure in one of the side vessels BV1, BV2, BV3.

Because the functional layer initially reduces the blood flow in a side vessel BV1, BV2, BV3, a pressure drop or a strong pressure drop is generated between the blood pressure in the main vessel MV and the corresponding side vessel BV1, BV2, BV3. This pressure drop or this pressure gradient generates a force, which is sufficiently high to expand the pores of the functional layer 4. This occurs because the filaments of the functional layer 4 are elastically and/or plastically deformed and/or slide on one another, so that exclusively in the region of the branching blood vessel, i.e. locally in the region of the opening into the corresponding side vessel BV1, BV2, BV3, the functional layer 4 becomes permeable to blood or more permeable to blood. The membrane 2 is “intelligent” insofar as it only allows blood to flow through at those sites at which the pressure gradient between the blood pressure in the main vessel MV and a pressure outside the outer membrane 2 is sufficiently high. This threshold is regularly exceeded at sites of the membrane 2, which cover the side vessels BV1, BV2, BV3 which branch off the main vessel MV. At the site on the membrane 2 which bridges the aneurysm AN which opens from the main vessel MV, the pressure threshold is not exceeded, i.e. the pressure gradient between the blood pressure in the main vessel MV and a pressure inside the aneurysm AN is not sufficiently large to expand the pores of the functional layer 4. Thus, the aneurysm AN remains shielded from the bloodstream, so that blood remaining in the aneurysm AN coagulates within a short period and the aneurysm AN therefore atrophies.

If an embolization means 30 is additionally arranged in the aneurysm, as can be seen in the exemplary embodiment of FIG. 1, then the support layer 3, which essentially has a stabilizing function, also serves to retain the embolization means 30 in the aneurysm AN, which therefore does not move back into the main vessel MV. This additionally ensures that the aneurysm AN atrophies in a timely manner.

FIGS. 2 and 3 respectively show a section of the implant with a carrier structure 1 and a membrane 2. The carrier structure 1 is formed by a mesh structure 10, wherein in FIGS. 2 and 3, several webs 12 of the mesh structure 10 are respectively indicated. The webs 12 form cells 13 of the mesh structure 10. In the exemplary embodiment, which is depicted, the webs 12 are monolithically interconnected. As a consequence, the mesh structure 10 is formed as one piece. However, it is also possible for the mesh structure 10 to be formed by wires, which are intertwined or interwoven.

The cell 13 is bridged by the membrane 2. The membrane 2 comprises at least two layers, which each are formed by electrospun filaments. The layers differ in their thickness and the density of the filaments.

Specifically, the membrane 2 has a support layer 3, which has a relatively lower density of filaments with a relatively higher filament thickness. The support layer 3 therefore differs from a functional layer 4, the filaments of which have a smaller filament thickness. Furthermore, the density of the filaments of the functional layer 4 is higher than the density of the filaments of the support layer 3. In other words, the support layer 3 and the functional layer 4 have pores 5 which are respectively delimited by the filaments and which are larger in the support layer 3 than in the functional layer 4. This is in any case true for the rest state of the implant, i.e. without any external force being exerted.

The functional layer 4 is tasked with impeding or at least slowing down the flow of blood through the membrane 2. In this regard, the functional layer 4 works like a flow diverter, i.e. deflecting the flow of blood along its surface. Because of the small filament thickness, the functional layer is relatively flexible. The support layer 3 stabilizes the functional layer 4 and prevents the functional layer 4 from bulging out in the radial direction, or ensures that the functional layer 4 lies tightly against the carrier structure 1.

FIG. 2 shows the principle of deflection of the blood flow. The membrane 2, which extends over the cell 13, bridges an aneurysm AN. Between the aneurysm AN and the main vessel MV into which the implant has been inserted there is barely any relevant pressure gradient, so that the functional layer 4 remains substantially in its passive state. As a consequence, the functional layer 4 has a small pore size, so that the blood flow is guided mainly along the functional layer 4 and essentially does not penetrate into the aneurysm AN. Thus, the aneurysm AN is substantially uncoupled from the blood flow in the main vessel MV and can atrophy by coagulation of the blood remaining in the aneurysm AN. Nevertheless, a small flow of blood can flow into the aneurysm through the pores of the membrane 2, so that the coagulation process and the formation of a solid thrombus in the aneurysm is not interrupted.

FIG. 3 shows the function of the functional layer 4 when it bridges a branching blood vessel, for example the second side vessel BV2. Because of the pressure difference which arises between the main vessel MV and the second side vessel BV2, the filaments of the functional layer 4 are deflected or locally deformed. This causes the pores of the functional layer 4 to become enlarged in the region of the mouth of the second side vessel BV2. In contrast, the filaments of the support layer 3 are more stable and largely retain their position. The pores of the support layer 3, however, are still large enough to permit blood to flow through the support layer 3. This is sufficient for the pores of the functional layer 4 to become enlarged in the region of the mouth of the second side vessel BV2 in order to permit a sufficient flow of blood from the main vessel MV into the second side vessel BV2.

Various exemplary embodiments of medical implants wherein the functional layer 4 is provided with a perforation 14 are shown in FIGS. 4 to 7. For the purposes of clarity, the support layer 3 is not shown in FIGS. 4 to 7.

Specifically, FIGS. 4 to 7 respectively show a stent with a carrier structure 1 which is configured as a mesh structure 10. The mesh structure 10 comprises a plurality of webs 12 coupled together into one piece which delimit cells 13. Radiographic markers 11 are arranged at the longitudinal ends of the mesh structure 10. A membrane 2 with a functional layer 4 is provided in a central region of the mesh structure 10. The membrane 2 extends over the entire circumference of the mesh structure 10 and completely covers the cells 13. The membrane 2 is connected to the webs 12 of the mesh structure 10.

The functional layer 4 depicted in FIGS. 4 to 7 has a perforation 14. The perforation 14 is preferably distributed in a pattern over the functional layer 4. In particular, the perforation 14 lies in the region of mesh openings or cells 13 of the carrier structure 1. With regard to the patterned arrangement of the perforation 14 there are commonalities in the exemplary embodiments of FIGS. 4 to 7. Thus, in the exemplary embodiments, which are depicted, the density of the perforations 14 in cells 13 which are arranged close to the longitudinal end of the functional layer 4 is higher than in cells 13 of the central region of the functional layer 4. When positioning the implant in the region of an aneurysm AN, it should be ensured that the flow of blood into the aneurysm AN is interrupted to a certain extent. A local opening of the functional layer 4 in the region of the aneurysm AN is therefore undesirable. Usually, the implant is positioned in a manner such that the central region of the implant, in particular of the functional layer 4, is placed in the region of the aneurysm AN. The fact that perforations 14 are still present in this region, albeit in a lower density, means that the aneurysm AN, for example, could still be well supplied with nutrients compared with branched side vessels BV1, BV2, BV3, because the perforation 14 provides an opening in the functional layer 4. The probability that a side vessel BV1, BV2, BV3 will be covered by the functional layer 4 is higher in the edge regions thereof. The perforation 14 provided there is more permeable.

The exemplary embodiments 4 to 7 differ in the type of perforation 14 in the functional layer 4. Thus, FIG. 4 shows an exemplary embodiment in which the functional layer 4 has a perforation 14 formed by holes 14a. The holes 14a are substantially in regions of the functional layer 4 which cover the mesh openings or cells 13. The number of holes 14a in the cells 13, which are arranged at the longitudinal ends of the functional layer 4 is greater than in a central region of the functional layer 4.

In the exemplary embodiment in accordance with FIG. 5, the perforations 14 are formed by straight slits 14b, which extend parallel to the longitudinal axis of the mesh structure 10. A different orientation of the straight slits 14b is possible. In particular, the straight slits 14b may be arranged at an angle of between 0° and 180° with respect to a longitudinal axis of the implant projected into the plane of the wall of the implant.

In the exemplary embodiment in accordance with FIG. 5, the length of the slits 14b is adjusted to the space, which is available between neighbouring webs 12 in the longitudinal direction of the mesh structure 10, so that the slits 14b are of different lengths. The spacing of the slits 14b in the circumferential direction of the mesh structure 10 also varies, wherein in edge regions of the functional layer 4, the spacing is smaller than in a central region of the functional layer 4.

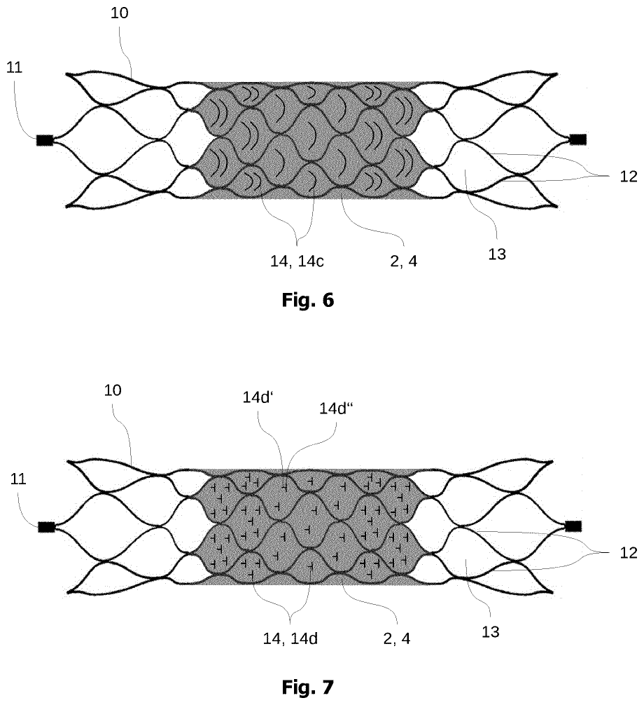

The exemplary embodiment in accordance with FIG. 6 shows an implant with a functional layer 4, which has a perforation 14 formed by curved slits 14c. The curved slits 14c extend substantially in the circumferential direction of the mesh structure 10. Two curved slits 14c are arranged in each cell 13 in the edge regions of the functional layer 4, whereas in a central region of the functional layer 4, each cell 13 is associated with one curved slit 14c. A different number and distribution of the perforations 14 is possible.

The curved slits 14c are preferably orientated in the same direction and in particular in the direction of flow of the blood. In other words, the implant in accordance with FIG. 6 is preferably placed in a blood vessel in a manner such that the blood flows from the longitudinal ends of the curved slits 14c to the apex of its curvature. In the representation of FIG. 6, therefore, the blood would flow from the left end of the mesh structure 10 to the right end of the mesh structure 10. The curved slits 14c therefore form gill-like openings in the functional layer 4.

In general, for all of the exemplary embodiments in which a perforation 14 is provided, the support layer 3 has a restraining function for the opening of the perforation 14. Particularly in the case of the gill-like embodiment of the openings, the perforation 14 opens by deflection of a portion of the functional layer 4. This deflection is limited by the support layer 3, which in this regard has a restraining function for the valve-like opening of the perforation 14. The restraining function of the support layer 3 and the perforation 14 of the functional layer 4 are therefore matched in a manner such that the perforation 14 then only opens when a predetermined pressure gradient exists between the inside of the membrane 2 and the outside of the membrane 2.

FIG. 7 shows an exemplary embodiment in which the perforation 14 of the functional layer 4 is formed by T-shaped slits 14d. In this exemplary embodiment as well, more T-shaped slits 14d per cell 13 are provided in the edge regions of the functional layer 4 than in a central region of the functional layer 4. The T-shaped slits 14d are preferably orientated in the same direction. In particular, each T-shaped slit 14d comprises a main slit 14d′ and a cross-slit 14d″, wherein the main slit 14d′ extends parallel to the longitudinal axis of the mesh structure 10 and the cross-slit 14d″ extends perpendicular thereto. The cross-slit 14d″ connects to a distal longitudinal end of the main slit 14d′. Preferably, the implant is placed in the blood vessel in a manner such that blood flows from the proximal end of the main slit 14d′ to the cross-slit 14d″.

### LIST OF REFERENCE NUMERALS

1 carrier structure
2 membrane
3 support layer
4 functional layer
5 pore
10 mesh structure
11 radiographic marker
12 web
13 cell
14 perforation
14a hole
14b straight slit
14c curved slit
14d T-shaped slit
14d′ main slit
14d″ cross-slit
embolization means
AN aneurysm
BV1 first side vessel
BV2 second side vessel
BV3 third side vessel
MV main vessel

1-18. (canceled)

19. A medical implant for treatment of an aneurysm comprising:
a carrier structure having a compressible and expandable mesh structure with mesh elements configured to delimit mesh openings, wherein the mesh structure is covered, at least in one or more sections, with a membrane of fibres including at least one luminal functional layer and at least one abluminal support layer, each layer respectively having pores, wherein a porosity of the functional layer is smaller than the porosity of the support layer, and
wherein the membrane is configured such that, as a consequence of a pressure gradient occurring between a first liquid pressure in an inner through channel of the carrier structure and a second liquid pressure outside the support layer, at least the pores of the functional layer open to increase a flow of liquid through the membrane.

20. The medical implant according to claim 19, wherein the fibres of the membrane are arranged loosely on top of one another at points of intersection, so that intersecting fibres are movable with respect to each other at the points of intersection, and wherein at least the fibres of the functional layer of the membrane are elastically or plastically deformable.

21. The medical implant according to claim 19, wherein the fibres of the functional layer of the membrane have a fibre thickness of less than 500 nm and wherein the fibres of the support layer of the membrane have a fibre thickness of at least 500 nm.

22. The medical implant according to claim 19, wherein the functional layer of the membrane has a thickness of less than 10 μm and wherein the support layer of the membrane has a thickness of at least 3 μm.

23. The medical implant according to claim 19, wherein the functional layer of the membrane has a porosity of less than 50% and wherein the support layer of the membrane has a porosity of at least 50%.

24. The medical implant according to claim 19, wherein the functional layer of the membrane comprises at least 10 pores having an inscribed circle diameter of at most 10 μm over a surface area of 100000 μm², and wherein the support layer of the membrane comprises at least 5 pores having an inscribed circle diameter of at least 10 μm over a surface area of 100000 μm².

25. The medical implant according to claim 19, wherein the fibres of the functional layer have a smaller fibre thickness than the fibres of the support layer, and wherein the functional layer has a higher ductility than the support layer or its fibres.

26. The medical implant according to claim 19, wherein the fibres of the functional layer are formed from a material which has a lower Shore hardness than the material of the fibres of the support layer.

27. The medical implant according to claim 26, wherein the material of the fibres of the functional layer has a Shore hardness of at most 90A and wherein the material of the fibres of the support layer has a Shore hardness of at least 90A.

28. The medical implant according to claim 19, wherein the membrane comprises a thermoplastic polyurethane.

29. The medical implant according to claim 19, wherein the membrane extends around an entire circumference of the carrier structure.

30. The medical implant according to claim 19, wherein the carrier structure is monolithic in configuration, and wherein the mesh elements of the mesh structure form webs configured to delimit the mesh openings of the mesh structure which are formed as cells.

31. The medical implant according to claim 19, wherein the carrier structure has interwoven wires, and wherein the wires form the mesh elements of the mesh structure and delimit the mesh openings of the mesh structure which are formed as interstices.

32. The medical implant according to claim 31, wherein the membrane has a total layer thickness which is at most 40% of a height of the mesh elements.

33. The medical implant according to claim 31, wherein a height of the mesh elements is between 40 μm and 160 μm.

34. The medical implant according to claim 31, wherein a ratio between a thickness of the membrane and a height of the mesh elements is at most 1/3.

35. The medical implant according to claim 19, wherein the functional layer has a perforation in a region of the mesh openings.

36. The medical implant according to claim 35, wherein the perforation is formed by one of holes, straight slits, curved slits, or T-shaped slits.

37. A method for production of a medical implant, the method comprising:
providing a carrier structure having a compressible and expandable mesh structure with mesh elements configured to delimit mesh openings;
applying a luminal functional layer of a membrane of fibres to the carrier structure;
perforating the functional layer by one of a laser cutting process or solvent spraying; and
applying an abluminal support layer of the membrane to the functional layer.
